(19) United States
(12) Patent Application Publication
(10) Pub. No.: US 2026/0251540 A1
(43) Pub. Date: Aug. 27, 2026

(54) Compositions and Methods for Whole Blood Preservation

(71) Applicant: ImYoo Inc., South San Francisco, CA (US)

(72) Inventors: Jong Hwee Park, South San Francisco, CA (US); Tatyana Dobreva, South San Francisco, CA (US); David Brown, South San Francisco, CA (US)

(73) Assignee: ImYoo Inc., South San Francisco, CA (US)

(21) Appl. No.: 19/162,876

(22) PCT Filed: Mar. 6, 2024
(86) PCT No.: PCT/US2024/018734

§ 371 Date: Sep. 5, 2025

Related U.S. Application Data
(60) Provisional application No. 63/489,172, filed on Mar. 8, 2023.

(51) Int. Cl. G01N 1/30

(52) U.S. Cl. CPC G01N 1/30

(57) ABSTRACT

Systems and methods for small volume whole blood preservation are provided. Whole blood can be preserved Start and stored for an extended period of time at room temperature. The preserved whole blood can be used in single cell-based applications.

### CROSS REFERENCE TO RELATED APPLICATIONS

The current application claims the benefit and priority of U.S. Provisional Patent Application No. 63/489,172 entitled “Compositions and Methods for Whole Blood Preservation” filed Mar. 8, 2023. The disclosure of U.S. Provisional Patent Application No. 63/489,172 is incorporated by reference in its entirety for all purposes.

### TECHNICAL FIELD

The current disclosure is directed to compositions and methods for preserving whole blood; and more particularly to compositions and methods for preserving whole blood for single cell nucleic acid processing.

### BACKGROUND

Whole blood contains red blood cells (erythrocytes), white blood cells (leukocytes), and platelets (thrombocytes) suspended in blood plasma. Blood cells contain various biomolecules such as proteins and nucleic acids. Proteins such as hemoglobin in red blood cells carry oxygen from the lungs to all parts of the body. Many tests are designed to determine the number of erythrocytes and leukocytes in the blood, together with the volume, sedimentation rate, and hemoglobin concentration of the red blood cells (blood count). In addition, certain tests are used to classify blood according to specific red blood cell antigens, or blood groups. Other tests may elucidate the shape and structural details of blood cells and hemoglobin and other blood proteins. Blood also can be analyzed to determine the activity of various enzymes, or protein catalysts, that either are associated with the blood cells or are found free in the blood plasma.

Blood may also be analyzed on the basis of properties such as total volume, circulation time, viscosity, clotting time and clotting abnormalities, acidity (pH), levels of oxygen and carbon dioxide, and the clearance rate of various substances. There are also tests based on the presence in the blood of substances characteristic of specific infections, such as the serological tests for syphilis, hepatitis, and human immunodeficiency virus (HIV).

Whole blood is typically stored at cryogenic temperature to preserve the biomolecules for downstream applications. However, as cells undergo the cooling process for cryogenic storage, their gene expression can alter, resulting in a different expression state post-extraction. Furthermore, prolonged exposure to cold temperatures will result in cell death. Cold storage can also incur extra cost in supplies and complex logistics. Methods to preserve whole blood without the use of cold storage and shipping can broaden blood-based analysis technologies to decentralized consumers.

### BRIEF SUMMARY

Compositions and methods for preserving whole blood and the components thereof are described.

Some embodiments of the invention include a method to preserve whole blood, comprising contacting a whole blood sample with a solution comprising a saccharide-epichlorohydrin copolymer and a molecular compound comprising an aldehyde functional group to yield a whole blood solution, and wherein a ratio (v/v) of the solution to the whole blood sample is equal to or greater than 20; and adding a quencher to the whole blood solution.

In some embodiments, the whole blood sample has a volume from 20 μL to 1 mL.

In some embodiments, the whole blood sample is collected using a capillary blood collection procedure.

In some embodiments, the molecular compound comprising the aldehyde functional group is: formaldehyde, paraformaldehyde, acetaldehyde, glutaraldehyde, or formalin.

In some embodiments, the molecular compound comprising an aldehyde functional group has a concentration ranging from 0.5% to 5.0% (% w/v).

In some embodiments, the saccharide-epichlorohydrin copolymer has a molecular weight from 60,000 Da to 500,000 Da.

In some embodiments, the saccharide-epichlorohydrin copolymer is sucrose-epichlorohydrin copolymer.

In some embodiments, the sucrose-epichlorohydrin copolymer is selected from the group consisting of: polysucrose, polysucrose 400, 2-(chloromethyl)oxirane; (2R,3R,4S,5S,6R)-2-[(2S,3S,4S,5R)-3,4-dihydroxy-2,5-bis(hydroxymethyl)oxolan-2-yl]oxy-6- (hydroxymethyl)oxane-3,4,5-triol, Ficoll®, Ficoll® 70, and Ficoll® 400.

In some embodiments, the saccharide-epichlorohydrin copolymer has a concentration from 2% to 20% (% w/v).

In some embodiments, the solution is dissolved in a solution selected from the group consisting of: distilled water, deionized water, phosphate buffer, phosphate buffer solution, saline, HEPES buffer, MES buffer, MOPS buffer, PIPES buffer, potassium phosphate, sodium phosphate, TAPS, citric acid—sodium citrate buffer, bicarbonate buffer, and any combinations thereof.

In some embodiments, the contacting step is performed at a temperature from 20 ° C. to 25° C.

In some embodiments, the quenching step is performed 20 minutes to 60 minutes after the contacting step is performed.

In some embodiments, the quencher comprises a molecular compound comprising an amine functional group, dissolved in a solution comprising a sucrose-epichlorohydrin copolymer.

In some embodiments, the molecular compound comprising an amine functional group is provided as a tris buffer, a glycine buffer, or a combination thereof; wherein the tris buffer comprises a tris base selected from the group consisting of: tris(hydroxymethyl)aminomethane, Trizma® base, amino-2-(hydroxymethyl)-1,3-propanediol, 2-,2-Amino-2-(hydroxymethyl)-1,3-propanediol, THAM, tris(hydroxymethyl)aminomethane, and trometamol buffer.

In some embodiments, the quencher has an equivalent or excess molar concentration to the molecular compound comprising the aldehyde functional group.

In some embodiments, the quencher is provided in a quenching solution in a ratio (v/v) to the solution equal to or greater than 2.

In some embodiments, the quencher completes quenching in a time duration from 5 minutes to 60 minutes.

Some embodiments include a method to preserve whole blood for single cell applications, comprising:
contacting a whole blood sample with a solution comprising a saccharide-epichlorohydrin copolymer and a molecular compound comprising an aldehyde functional group to yield a whole blood solution, wherein a ratio (v/v) of the fixation solution to the whole blood sample is equal to or greater than 20;
adding a quencher to the whole blood solution;
enriching nucleated cells from the whole blood solution; and
performing a single cell assay on the enriched nucleated cells.

In some embodiments, the whole blood sample has a volume from 20 μL to 1 mL.

In some embodiments, the whole blood sample is collected using a capillary blood collection procedure.

In some embodiments, the molecular compound comprising the aldehyde functional group is: formaldehyde, paraformaldehyde, acetaldehyde, glutaraldehyde, or formalin.

In some embodiments, the molecular compound comprising an aldehyde functional group has a concentration ranging from 0.5% to 5.0% (% w/v).

In some embodiments, the saccharide-epichlorohydrin copolymer has a molecular weight from 60,000 Da to 500,000 Da.

In some embodiments, the saccharide-epichlorohydrin copolymer is sucrose-epichlorohydrin copolymer.

In some embodiments, the sucrose-epichlorohydrin copolymer is selected from the group consisting of: polysucrose, polysucrose 400, 2-(chloromethyl)oxirane; (2R,3R,4S,5S,6R)-2-[(2S,3S,4S,5R)-3,4-dihydroxy-2,5-bis(hydroxymethyl)oxolan-2-yl]oxy-6- (hydroxymethyl)oxane-3,4,5-triol, Ficoll®, Ficoll® 70, and Ficoll® 400.

In some embodiments, the saccharide-epichlorohydrin copolymer has a concentration from 2% to 20% (% w/v).

In some embodiments, the solution is dissolved in a solution selected from the group consisting of: distilled water, deionized water, phosphate buffer, phosphate buffer solution, saline, HEPES buffer, MES buffer, MOPS buffer, PIPES buffer, potassium phosphate, sodium phosphate, TAPS, citric acid—sodium citrate buffer, bicarbonate buffer, and any combinations thereof.

In some embodiments, the contacting step is performed at a temperature from 20° C. to 25° C.

In some embodiments, the quenching step is performed 20 minutes to 60 minutes after the contacting step.

In some embodiments, the quencher comprises a molecular compound comprising an amine functional group, dissolved in a solution comprising sucrose-epichlorohydrin copolymer.

In some embodiments, the molecular compound comprising an amine functional group is provided as a tris buffer, a glycine buffer, or a combination thereof; wherein the tris buffer comprises a tris base selected from the group consisting of: tris(hydroxymethyl)aminomethane, Trizma® base, amino-2-(hydroxymethyl)-1,3-propanediol, 2-,2-Amino-2-(hydroxymethyl)-1,3-propanediol, THAM, tris(hydroxymethyl)aminomethane, and trometamol buffer.

In some embodiments, the quencher has an equivalent or excess molar concentration to the molecular compound comprising the aldehyde functional group.

In some embodiments, the quencher is provided in a quenching solution in a ratio (v/v) to the solution equal to or greater than 2.

In some embodiments, the quencher completes quenching in a time duration from 5 minutes to 60 minutes.

Some embodiments further comprise storing the quenched whole blood solution at a temperature from 20° C. to 25° C. for at least 24 hours.

In some embodiments, the nucleated cells comprise PBMCs.

Some embodiments further comprise isolating PBMCs from the quenched whole blood solution using a method selected from the group consisting of: centrifugation, density gradient centrifugation, a cell preparation tube, a PBMC isolation tubes, a magnetic cell separation kit, immunoprecipitation, immune separation, and column purification.

Some embodiments further comprise storing the isolated PBMCs in a freezer or using a cryogenic liquid.

In some embodiments, a single cell assay comprises single cell RNA sequencing (scRNA-seq).

In some embodiments, the scRNA-seq is carried out on a 10× Genomics® scRNA-seq platform.

Some embodiments include a solution for preserving whole blood, comprising a saccharide-epichlorohydrin copolymer; and a molecular compound comprising an aldehyde functional group; dissolved in a solution.

In some embodiments, the molecular compound comprising an aldehyde functional group is of: formaldehyde, paraformaldehyde, acetaldehyde, glutaraldehyde, or formalin.

In some embodiments, the molecular compound comprising an aldehyde functional group has a concentration ranging from 0.5% to 5.0% (% w/v).

In some embodiments, the saccharide-epichlorohydrin copolymer has a molecular weight from 60,000 Da to 500,000 Da.

In some embodiments, the saccharide-epichlorohydrin copolymer is sucrose-epichlorohydrin copolymer.

In some embodiments, the sucrose-epichlorohydrin copolymer is selected from the group consisting of: polysucrose, polysucrose 400, 2-(chloromethyl)oxirane; (2R,3R,4S,5S,6R)-2-[(2S,3S,4S,5R)-3,4-dihydroxy-2,5-bis(hydroxymethyl)oxolan-2-yl]oxy-6- (hydroxymethyl)oxane-3,4,5-triol, Ficoll®, Ficoll® 70, and Ficoll® 400.

In some embodiments, the saccharide-epichlorohydrin copolymer has a concentration from 2% to 20% (% w/v).

In some embodiments, the solution is selected from the group consisting of: distilled water, deionized water, phosphate buffer, phosphate buffer solution, saline, HEPES buffer, MES buffer, MOPS buffer, PIPES buffer, potassium phosphate, sodium phosphate, TAPS, citric acid—sodium citrate buffer, bicarbonate buffer, and any combination thereof.

Additional embodiments and features are set forth in part in the description that follows, and in part will become apparent to those skilled in the art upon examination of the specification or may be learned by the practice of the disclosed subject matter. A further understanding of the nature and advantages of the present disclosure may be realized by reference to the remaining portions of the specification and the drawings, which form part of this disclosure.

### BRIEF DESCRIPTION OF THE DRAWINGS

The description will be more fully understood with reference to the following figures, which are presented as example embodiments of the invention and should not be construed as a complete recitation of the scope of the invention, wherein:

FIG. 1 illustrates a process of preserving whole blood in accordance with an embodiment.

FIG. 2 illustrates a process of preserving whole blood for single cell analysis in accordance with an embodiment.

FIG. 3 illustrates various types of cells from whole blood preserved using the preservation process in accordance with an embodiment.

FIG. 4 illustrates a comparison of PBMCs preservation using an aldehyde solution and the preservation solution in accordance with an embodiment.

FIG. 5 illustrates MALAT1 gene expression of isolated cells from preserved whole blood at different time points in accordance with an embodiment.

FIG. 6 illustrates unique molecular identifiers counts of isolated cells from preserved whole blood at different time points in accordance with an embodiment.

FIG. 7 illustrates median UMI counts of various types of cells in accordance with an embodiment.

FIG. 8 illustrates a gene abundance correlation for CD4 T cells between 24 hours and 72 hours in accordance with an embodiment.

FIG. 9 illustrates a gene abundance correlation for CD8 T cells between 24 hours and 72 hours in accordance with an embodiment.

FIG. 10 illustrates a gene abundance correlation for CD14 monocytes between 24 hours and 72 hours in accordance with an embodiment.

FIG. 11 illustrates a gene abundance correlation for CD16 monocytes between 24 hours and 72 hours in accordance with an embodiment.

FIG. 12 illustrates a gene abundance correlation for NK cells between 24 hours and 72 hours in accordance with an embodiment.

FIG. 13 illustrates a gene abundance correlation for B cells between 24 hours and 72 hours in accordance with an embodiment.

FIG. 14 illustrates a correlation of percentage of cells for CD4 T cells, CD8 T cells, NK cells, B cells, and monocytes between 24 hours and 72 hours in accordance with an embodiment.

### TERMINOLOGY

The following definitions are provided to aid those skilled in the art in understanding the detailed description:

The phrase a “peripheral blood mononuclear cell (PBMC)” refers to any peripheral blood cell having a round nucleus. The PBMCs consist of lymphocytes (T cells (CD4+, CD8+), B cells, Natural Killer cells) and monocytes (CD14+, CD16+). Erythrocytes and platelets have no nuclei, and granulocytes (neutrophils, basophils, and eosinophils) have multi-lobed nuclei.

The phrase “capillary blood” refers to a blood sample that is collected by pricking the skin of a finger, a heel, the deltoid, or another area of a body with a sharp needle or a lancet. Capillary blood is not obtained from a vein or by venipuncture.

The phrase “preservation” refers to the preservation of biological samples from decay by terminating any ongoing biochemical reactions.

The phrase “preservative” and “preservation agent” refer to chemicals and formulations used during the preservation process.

The phrase “RNA sequencing (RNA-seq)” refers to a sequencing technique which uses next-generation sequencing (NGS) to reveal the quantity of RNA and gene expression in a biological sample.

The phrase “room temperature” is used interchangeably as ambient temperature, and refers to a temperature between about 20° C. to about 25° C. (68° F. to 77° F.), with excursions between about 15° C. to about 30° C. (59° F. to 86° F.).

The phrase “aldehyde” refers to an organic compound containing a functional group with the structure R—CH═O, where R is a side chain.

### DETAILED DESCRIPTION

Turning to the drawings, descriptions of systems and methods for whole blood preservation for single cell applications are provided. An individual's immune system is driven by both genetic and environmental factors that vary over time. Single cell analysis platforms such as multiplexed single cell RNA sequencing (scRNA-seq) utilizing blood has been developed to better understand the temporal and inter-individual variability of gene expression within distinct immune cell types. For instance, performing scRNA-seq on capillary blood can identify genes that exhibit diurnal behavior in subpopulations of cells and find subject-specific immune relevant gene signatures. (See, e.g., U.S. Patent Publication No. 2021/0324447 to M. W. Thomson, et al., the disclosure of which is incorporated by reference in its entirety.)

Small volume blood is being increasingly used in point-of-care testing because only a small amount of sample is required. Capillary blood sampling is typically used for collecting small volume blood due to its simplified and cost-effective collection process compared to venipuncture. Although out-of-clinic capillary blood extraction eases patient burden physically and financially, preserving and transporting a small volume of whole blood requires storage at cold temperatures (such as from about −10° C. to about 10° C.) and fast turnaround in order to preserve gene expression information. Blood left sitting out at room temperature and even at cold temperature over a prolonged time period can cause the cells to change their gene expression. Single-cell gene expression and high-plex plasma proteomics provide evidence that cells exhibit time-dependent changes that distort the underlying biology. These changes are broad and dynamic, complicating the technical analysis of scRNA-seq data and inferences of in vivo physiology from ex vivo assays. (See, e.g., A. K. Savage, et al., iScience, 24, 5, 102404, 2021; the disclosure of which is incorporated herein by reference.) This change of gene expression and tendency towards apoptosis can shift cell type distributions, masking the real biological signal intended to be captured. Attempts at preserving immune cells have been limited to hospitals and mobile phlebotomy, constrained by expensive costs of cold shipping and personnel logistics.

Various systems and methods for preserving whole blood in accordance with many embodiments of the disclosure can preserve whole blood inclusive of the cells and biomolecules thereof, for a period of time at room temperature. The preservation processes can preserve cells and other components from whole blood including (but not limited to) nucleated cells, red blood cells (erythrocytes), platelets, pathogens, and biomolecules including (but not limited to) nucleic acids (DNA and RNA), proteins, polysaccharides, and lipids, while reducing and/or minimizing artifacts during the duration of storage. In several embodiments, whole blood is fixated at a particular time (e.g., at or near extraction) such that the biological activities of the blood cells are preserved from that fixed timepoint. In some instances, the preserved whole blood can be used immediately after fixation (e.g., within 1 hour). In some embodiments, the preserved whole blood can be stored for an extended time period at room temperature (e.g., at least 24 hours). In some instances, the storage of preserved whole blood can be carried out at room temperature without additional steps such as heating or cooling. The procedures of collecting, preserving, and storing whole blood at room temperature, are easy to follow such that decentralized users can self-collect the blood sample, safely and accurately carry out the preservation processes, and properly store and/or return the blood sample.

Various systems and methods preserve nucleated cells in whole blood for downstream single cell analysis. The preserved nucleated cells can be stored at room temperature for an extended period of time before being performed single cell applications. Analysis of genomics, transcriptomics, proteomics, metabolomics, and cell-cell interactions at a single cell level can help elucidate cell-to-cell variation and identify unique cells within a cell population. In several embodiments, preservation of whole blood allows for analysis of biomolecules including (but not limited to) nucleic acids, proteins, and metabolic compounds within nucleated cells in a variety of single cell applications. Examples of single cell assays that are compatible with the preserved whole blood include (but are not limited to): single cell nucleic acid sequencing, single cell DNA sequencing, single cell genomic analysis, single cell RNA sequencing, single cell transcriptome analysis, single cell gene expression assays, single cell proteomic analysis (e.g., via mass spectrometry), single cell immunodetection assays, single cell metabolomic analysis (e.g., via mass spectrometry), single cell chromatin analysis, single cell methylome sequencing, single cell bisulfite sequencing, single cell immunoprecipitation assays, and single cell chromatin immunoprecipitation sequencing.

Certain embodiments preserve nucleated cells. Nucleated cells can include any nucleated cell that can be derived from whole blood and include cells with multilobed nuclei. Nucleated cells that can be preserved include (but are not limited to) peripheral blood monocytes (PBMCs), granulocytes, blood cancer cells, circulating tumor cells, and pathogens. PBMCs include (but are not limited to) lymphocytes, monocytes, T cells, B cells, natural killer cells, and dendritic cells. Granulocytes include (but are not limited to) neutrophils, basophils, and eosinophils. Blood cancer cells include (but are not limited to) leukemia cells, lymphoma cells, and myeloma cells. Circulating tumor cells are cells that have shed off of a tumor (primary or otherwise) into the blood stream and thus can originate from any solid tissue. Pathogens can include any pathogen that could be found in the blood stream (e.g., a bloodborne pathogens, bloodstream infections), including intracellular and extracellular pathogens. Examples of pathogens include (but are not limited to) hepatitis B virus (HBV), hepatitis C virus (HCV), human immunodeficiency virus (HIV), Staphylococcus aureus, Streptococcus pneumoniae, Escherichia coli, Plasmodium falciparum, Toxoplasma gondii, and Trypanosoma. Nucleated cells can be enriched or isolated from the whole blood for downstream applications. In one example, collected nucleated cells can be utilized for single cell RNA-seq for immune profiling.

Small volume blood refers to a whole blood sample with a volume equal to or less than about 1 mL. Small volume blood can be collected via capillary blood sampling, venipuncture sampling, and/or arterial sampling. Systems and methods for preserving capillary whole blood enable decentralized users to self-collect their capillary blood and send it to a facility for single-cell processing and analysis. The preservation methods in accordance with many embodiments can keep the biomolecules of the cells in a fixated and stable state for an extended period of time at ambient temperature. The preservation of whole blood at room temperature for an extended period of time can minimize the complex and costly logistics of cold shipping. The fixative and preservative solutions can sufficiently preserve biomolecules while reducing and/or minimizing artifacts during fixation, preservation, and storage.

### Cell Preservation

Storage and preservation of whole blood is important for biological research and clinical assessment to minimize contamination and genetic changes within the cells of the biological sample. Traditionally, cryopreservation, where cells and tissues are stored in a cryogenic environment, is utilized to preserve the isolated cells from whole blood. Standard cryopreservation techniques require that nucleated cells are isolated from whole blood prior to cryopreservation. Cell isolation, which typically require laboratory techniques such as gradient buffers and centrifugation, prevents decentralized users lacking the skill or access to a laboratory from preserving whole blood samples at home via cryopreservation. The ability to cryogenically preserve whole blood permits the transportation of biological samples, and extends the lifetime for completion of safety and quality control testing. Cryopreservation, however, typically requires: pre-freeze processing, introduction of a cryopreservation solution, and a cryogenic storage in order to carry out the freezing protocol. Cryogenic processes can be difficult and expensive to perform due to the costs and extreme conditions to keep and store liquid nitrogen.

Cell fixation preserves cellular morphology, integrity and structure by spatiotemporally fixing biomolecules and preventing the process of putrefaction (cellular decay). Biological samples can be fixed immediately following extraction to limit autolysis and putrefaction. Common cell fixation reagents include precipitating fixatives and cross-linking fixatives. Organic solvents, such as methanol, acetone, and picric acid, can act as strong dehydrants and precipitate cellular proteins. While these fixatives are effective at preserving cellular architecture, they can remove small soluble molecules and lipids.

Aldehydes, such as formalin, glutaraldehyde, paraformaldehyde or formaldehyde, are fixatives that crosslink proteins via free amine groups, forming intermolecular bridges. Biological samples can be incubated with a solution of formalin, glutaraldehyde, paraformaldehyde or formaldehyde at room temperature to perform the crosslinking reaction.

### Preservation of Whole Blood

Many embodiments of the disclosure are directed to various preservation processes to preserve small volumes of whole blood. The preservation processes can preserve biomolecules of single cells within whole blood for various downstream applications including (but not limited to) single cell nucleic acid sequencing, single cell DNA sequencing, single cell genomic analysis, single cell RNA sequencing, single cell transcriptome analysis, single cell gene expression assays, single cell proteomic analysis (e.g., via mass spectrometry), single cell immunodetection assays, single cell metabolomic analysis (e.g., via mass spectrometry), single cell chromatin analysis, single cell methylome sequencing, single s cell bisulfite sequencing, single cell immunoprecipitation assays, and single cell chromatin immunoprecipitation sequencing.

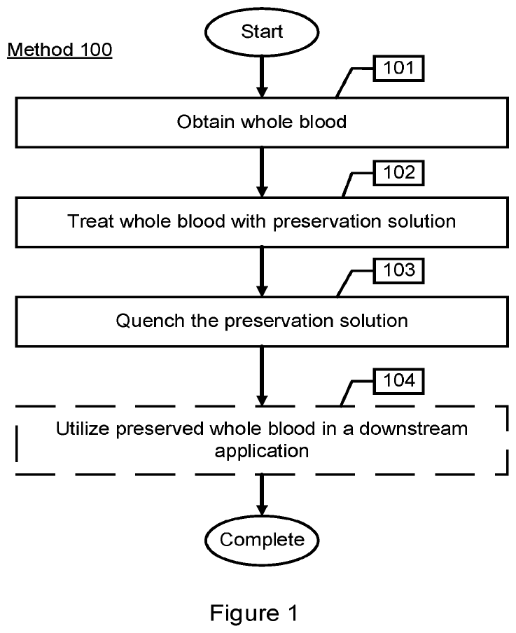

FIG. 1 illustrates a preservation process of whole blood in accordance with an embodiment of the disclosure. Method 100 can start with obtaining (101) a sample of small volume whole blood. The small volume of whole blood sample can be collected using any blood collection methodology, such as (for example) capillary blood sampling, venipuncture sampling, and/or arterial sampling. In one example, a sample of small volume whole blood can be collected via a capillary blood collection procedure. The capillary whole blood can be collected by pricking the skin of any body part, such as (for example) a finger, an arm, a leg, a heel, a toe, or the deltoid. Often, capillary sampling is performed on an area of skin with a high number of capillaries. Various methods can be used to collect the capillary whole blood including (but not limited to) needles, microneedles, and/or lancets. The blood can be collected in a container including (but not limited to) pipette, tubes, vials, or microcontainers. The containers can be made with a non-reactive material including (but not limited to) glass, plastic, hard plastic, resin, or polymer. Examples of commercially available containers for collecting capillary whole blood include (but are not limited to) TAP Touch Activated Phlebotomy® devices, TAP Blood Collection® devices, Tasso+® devices.

In some embodiments, a sample of small volume whole blood can be collected following a venipuncture procedure where blood is collected from a vein. Blood can be drawn from a vein from (but not limited to) the elbow or the hand. A needle can be inserted into the vein, and the blood is collected in an air-tight vial or a syringe. In some embodiments, a sample of small volume whole blood can be collected following an arterial sampling procedure. A small volume of the blood sample from the venipuncture sampling and/or the arterial sampling can be removed using various methods including (but not limited to) pipetting.

In some embodiments, a sample of small volume whole blood has a volume from about 20 μL to about 1 mL. In various embodiments, a small volume of whole blood is a volume from about 20 μL to about 30 μL; a small volume of whole blood is a volume from about 30 μL to about 40 μL; a small volume of whole blood is a volume from about 40 μL to about 50 μL; a small volume of whole blood is a volume from about 50 L to about 60 μL; a small volume of whole blood is a volume from about 60 μL to about 70 μL; a small volume of whole blood is a volume from about 70 μL to about 80 μL; a small volume of whole blood is a volume from about 80 μL to about 90 μL; a small volume of whole blood is a volume from about 90 μL to about 100 μL; a small volume of whole blood is a volume from about 100 μL to about 110 μL; a small volume of whole blood is a volume from about 110 μL to about 120 μL; a small volume of whole blood is a volume from about 120 μL to about 130 μL; a small volume of whole blood is a volume from about 130 μL to about 140 μL; a small volume of whole blood is a volume from about 140 μL to about 150 μL; a small volume of whole blood is a volume from about 150 μL to about 200 μL; a small volume of whole blood is a volume from about 200 μL to about 250 μL; a small volume of whole blood is a volume from about 250 μL to about 300 μL; a small volume of whole blood is a volume from about 300 μL to about 350 μL; a small volume of whole blood is a volume from about 350 μL to about 400 μL; a small volume of whole blood is a volume from about 400 μL to about 450 μL; or a small volume of whole blood is a volume from about 450 μL to about 500 μL; or a small volume of whole blood is a volume from about 500 μL to about 550 μL; or a small volume of whole blood is a volume from about 550 μL to about 600 μL; or a small volume of whole blood is a volume from about 600 μL to about 650 μL; or a small volume of whole blood is a volume from about 650 μL to about 700 μL; or a small volume of whole blood is a volume from about 700 μL to about 750 μL; or a small volume of whole blood is a volume from about 750 μL to about 800 μL; or a small volume of whole blood is a volume from about 800 μL to about 850 μL; or a small volume of whole blood is a volume from about 850 μL to about 900 μL; or a small volume of whole blood is a volume from about 900 μL to about 950 μL; or a small volume of whole blood is a volume from about 950 μL to about 1 mL.

The small volume whole blood sample can be treated (102) with a preservation solution. The preservation solution can fixate the whole blood and its constituents, preserving nucleated cells, biomolecules, or other constituents within whole blood for downstream analysis, especially analysis upon single cells. In many embodiments, the preservation solution comprises an aldehyde and a macromolecular crowder. Examples of macromolecular crowders include (but are not limited to) polyethylene glycol, dextran, and/or saccharide-epichlorohydrin copolymer. In many embodiments, the preservation solution comprises an aldehyde and a saccharide-epichlorohydrin copolymer (e.g., sucrose-epichlorohydrin copolymer).

The macromolecular crowder in the preservation solution can reduce cell aggregation during the time the cells are exposed to aldehyde so as to inhibit cell-to-cell contact to prevent cells from becoming fixed together. In embodiments utilizing a saccharide-epichlorohydrin copolymer, the saccharide can be any mono-, di-, oligo-, or poly-saccharide that can act as a macromolecular crowder when in a copolymer with epichlorohydrin. Examples of saccharide-epichlorohydrin copolymer that can be utilized include (but are not limited to) sucrose-epichlorohydrin copolymer. In one example of a sucrose-epichlorohydrin copolymer, an organic compound comprising a sucrose (of a molecular formula C₁₂H₂₂O₁₂)-epichlorohydrin (of a molecular formula C₃H₅ClO) copolymer can be used. In some embodiments, a sucrose-epichlorohydrin copolymer can have a molecular weight ranging from about 60,000 Da to about 500,000 Da. In various embodiments, a sucrose-epichlorohydrin copolymer has a molecular weight from about 60,000 Da to about 70,000 Da; a sucrose-epichlorohydrin copolymer has a molecular weight from about 70,000 Da to about 80,000 Da; a sucrose-epichlorohydrin copolymer has a molecular weight from about 80,000 Da to about 90,000 Da; a sucrose-epichlorohydrin copolymer has a molecular weight from about 90,000 Da to about 100,000 Da; a sucrose-epichlorohydrin copolymer has a molecular weight from about 100,000 Da to about 200,000 Da; a sucrose-epichlorohydrin copolymer has a molecular weight from about 200,000 Da to about 300,000 Da; a sucrose-epichlorohydrin copolymer has a molecular weight from about 300,000 Da to about 400,000 Da; or a sucrose-epichlorohydrin copolymer has a molecular weight from about 400,000 Da to about 500,000 Da. Examples of sucrose-epichlorohydrin copolymers include (but are not limited to): polysucrose, polysucrose 400, 2-(chloromethyl)oxirane; (2R,3R,4S,5S,6R)-2-[(2S,3S,4S,5R)-3,4-dihydroxy-2,5bis(hydroxymethyl)oxolan-2-yl]oxy-6-(hydroxymethyl)oxane-3,4,5-triol, Ficoll®, Ficoll® 70, and Ficoll® 400.

Various concentrations of sucrose-epichlorohydrin copolymer can be utilized in a preservation solution. In some embodiments, a sucrose-epichlorohydrin copolymer can have a concentration (% w/v) from about 2% to about 20%. In various embodiments, a sucrose-epichlorohydrin copolymer has a concentration from about 2% to about 3%; a sucrose-epichlorohydrin copolymer has a concentration from about 3% to about 4%; a sucrose-epichlorohydrin copolymer has a concentration from about 4% to about 5%; a sucrose-epichlorohydrin copolymer has a concentration from about 5% to about 6%; a sucrose-epichlorohydrin copolymer has a concentration from about 6% to about 7%; a sucrose-epichlorohydrin copolymer has a concentration from about 7% to about 8%; a sucrose-epichlorohydrin copolymer has a concentration from about 8% to about 9%; a sucrose-epichlorohydrin copolymer has a concentration from about 9% to about 10%; a sucrose-epichlorohydrin copolymer has a concentration from about 10% to about 11%; a sucrose-epichlorohydrin copolymer has a concentration from about 11% to about 12%; a sucrose-epichlorohydrin copolymer has a concentration from about 12% to about 13%; a sucrose-epichlorohydrin copolymer has a concentration from about 13% to about 14%; a sucrose-epichlorohydrin copolymer has a concentration from about 14% to about 15%; a sucrose-epichlorohydrin copolymer has a concentration from about 15% to about 16%; a sucrose-epichlorohydrin copolymer has a concentration from about 16% to about 17%; a sucrose-epichlorohydrin copolymer has a concentration from about 17% to about 18%; a sucrose-epichlorohydrin copolymer has a concentration from about 18% to about 19%; or a sucrose-epichlorohydrin copolymer has a concentration from about 19% to about 20%.

The preservation solution can comprise a molecular compound having an aldehyde functional group to fixate cells in whole blood. The aldehyde can crosslink biomolecules via free amine groups, forming intermolecular bridges and a network of linked molecules. Any organic compounds comprising an aldehyde functional group can be used for fixation in the preservation solutions in accordance with some embodiments. Examples of aldehyde compounds include (but are not limited to): formaldehyde, paraformaldehyde, acetaldehyde, glutaraldehyde, formalin, and combinations thereof. As can be readily appreciated, any of a variety of aldehyde compounds can be utilized in the preservation solution as appropriate to the requirements of specific applications in accordance with various embodiments of the invention.

The concentration of aldehyde molecules in a preservation solution can vary. Higher concentrations of aldehydes may increase the amount of crosslinking of biomolecules, which can better preserve the biomolecules but may hinder downstream applications. Thus, an appropriate concentration of aldehyde and the time of treatment can be optimized based on the needs of the specific downstream applications to be performed. Aldehyde can have a concentration (% w/v) from about 0.5% to about 5%; or from about 0.5% to about 1.0%; or from about 1.0% to about 1.5%; or from about 1.5% to about 2.0%; or from about 2.0% to about 2.5%; or from about 2.5% to about 3.0%; or from about 3.0% to about 3.5%; or from about 3.5% to about 4.0%; or from about 4.0% to about 4.5%; or from about 4.5% to about 5.0%.

In some embodiments, the preservation solution also comprises a permeabilization detergent. For example, Triton X-100, Tween-20, saponin or other permeabilizer can be utilized.

The preservation solution can be made utilizing an aqueous solution, such as (for example) water, deionized water, filtered water, or reverse osmosis water. In some embodiments, the preservation solution includes a buffer. Examples of buffers include (but are not limited to) phosphate buffer solution (PBS), HEPES buffer, MES buffer, MOPS buffer, PIPES buffer, potassium phosphate, sodium phosphate, TAPS, citric acid—sodium citrate buffer, bicarbonate buffer, saline, phosphate buffered saline, and any combinations thereof. The preservation solution can be preloaded in the containers for whole blood sample collection or can be added into the container containing the sample of collected whole blood. In some embodiments, the preservation solution is contacted with the whole blood sample having a large ratio of preservation solution to whole blood. In various embodiments, the ratio of preservation solution to whole blood is greater than or equal to about 10:1 (v/v), the ratio of preservation solution to whole blood is greater than or equal to about 15:1 (v/v), the ratio of preservation solution to whole blood is greater than or equal to about 20:1 (v/v), the ratio of preservation solution to whole blood is greater than or equal to about 25:1 (v/v), the ratio of preservation solution to whole blood is greater than or equal to about 30:1 (v/v), the ratio of preservation solution to whole blood is greater than or equal to about 35:1 (v/v), or the ratio of preservation solution to whole blood is greater than or equal to about 40:1 (v/v).

The contact time of the whole blood with the preservation solution can range from about 20 minutes to about 80 minutes. In various embodiments, the contact time of the whole blood with the preservation solution is from about 20 minutes to about 30 minutes; the contact time of the whole blood with the preservation solution is from about 30 minutes to about 40 minutes; the contact time of the whole blood with the preservation solution is from about 40 minutes to about 50 minutes; the contact time of the whole blood with the preservation solution is from about 50 minutes to about 60 minutes; the contact time of the whole blood with the preservation solution is from about 60 minutes to about 70 minutes; the contact time of the whole blood with the preservation solution is from about 70 minutes to about 80 minutes. In some embodiments, the contact time of the whole blood with the preservation solution is greater than or equal to about 45 minutes. In some embodiments, the contact time of the whole blood with the preservation solution is less than about 80 minutes. In some embodiments, the contact time of the whole blood with the preservation solution is from about 45 minutes to about 80 minutes. In certain embodiments, the contact time of the whole blood with the preservation solution of less than about 45 minutes may result in higher amount of cell debris, which may result in poor PBMC yield.

The contact of preservation solution with whole blood can be carried out at room temperature. The contact of preservation solution with whole blood can be carried out without heating, or heat treatment, or cooling, or cooling treatment.

When the preservation is complete, the preservation can be quenched (103) to inactivate the aldehyde 103. Contact with a quencher can allow extended storage of the preserved whole blood without removal of aldehydes from the whole blood solution. In some embodiments, a quencher is a molecular compound comprising an amine (or amino functional group). In some embodiments, the quencher is provided in a quenching solution; the quenching solution is used to contact the preservation solution to inactivate the aldehyde. In some embodiments, a quenching solution comprises a saccharide-epichlorohydrin copolymer including (but not limited to) a sucrose-epichlorohydrin copolymer and a quencher (e.g., molecular compound comprising an amine (or amino) functional group). The amine functional group can react with the aldehyde group and quench the crosslinking reaction. Examples of molecular compounds comprising an amine functional group include (but are not limited to): tris, tris buffer, glycine, glycine buffer, and any combinations thereof. Tris buffer includes a tris base dissolved in distilled water or deionized water with a pH ranging from about 6.0 to about 8.5; or from about 6.0 to about 6.5; or from about 6.5 to about 7.0; or from about 7.0 to about 7.5; or from about 7.5 to about 8.0; or from about 8.0 to about 8.5. Tris base is also known as tris(hydroxymethyl)aminomethane, Trizma® base, amino-2-(hydroxymethyl)-1,3-propanediol, 2-,2-Amino-2-(hydroxymethyl)-1,3-propanediol, THAM, tris(hydroxymethyl)aminomethane, and trometamol buffer. Tris glycine buffer includes a tris base and glycine dissolved in distilled water or deionized water with a pH ranging from about 6.0 to about 8.5; or from about 6.0 to about 6.5; or from about 6.5 to about 7.0; or from about 7.0 to about 7.5; or from about 7.5 to about 8.0; or from about 8.0 to about 8.5. The quenching solution can be dissolved in a buffer solution including (but not limited to) phosphate buffer solution, phosphate buffer, HEPES buffer, MES buffer, MOPS buffer, PIPES buffer, potassium phosphate, sodium phosphate, TAPS, citric acid—sodium citrate buffer, bicarbonate buffer, saline, phosphate buffered saline, water, deionized water, filtered water, and any combinations thereof.

In some embodiments, ethylenediaminetetraacetic acid (EDTA) can be added to the quenching buffer solutions. EDTA in the quenching buffer can provide additional protection against nuclease activities in accordance with certain embodiments. In some embodiments, the concentrations of EDTA in the quenching buffer can range from about 1 mM to about 10 mM; from about 1 mM to about 2 mM; or from about 2 mM to about 3 mM; or from about 3 mM to about 4 mM; or from about 4 mM to about 5 mM; or from about 5 mM to about 6 mM; or from about 6 mM to about 7 mM; or from about 7 mM to about 8 mM; or from about 8 mM to about 9 mM; or from about 9 mM to about 10 mM.

In some embodiments, the amine of the quencher has an equivalent molar concentration to the aldehyde in the fixation solution. In some embodiments, the amine of the quencher has an excess molar concentration to free aldehyde in the fixation solution. In certain embodiments, the ratio of glycine and aldehyde can have a molar ratio of about or greater than 1:2.

In some embodiments, a ratio of the quenching solution volume to the preservation solution volume is equal to or greater than or equal to about 2:1 (v/v); or a ratio of the quenching solution volume to the preservation solution volume is equal to or greater than or equal to about 3:1 (v/v); or a ratio of the quenching solution volume to the preservation solution volume is equal to or greater than or equal to about 4:1 (v/v); or a ratio of the quenching solution volume to the preservation solution volume is equal to or greater than or equal to about 5:1 (v/v); or a ratio of the quenching solution volume to the preservation solution volume is equal to or greater than or equal to about 6:1 (v/v), or a ratio of the quenching solution volume to the preservation solution volume is equal to or greater than or equal to about 7:1 (v/v), or a ratio of the quenching solution volume to the preservation solution volume is equal to or greater than or equal to about 8:1 (v/v), or a ratio of the quenching solution volume to the preservation solution volume is equal to or greater than or equal to about 9:1 (v/v), or a ratio of the quenching solution volume to the preservation solution volume is equal to or greater than or equal to about 10:1 (v/V).

The quenching time of the capillary whole blood with the quenching solution can range from about 60 minutes (about 1 hour) to about 240 minutes (about 4 hours); or from about 60 minutes to about 90 minutes; or from about 90 minutes to about 120 minutes; or from about 120 minutes to about 150 minutes; or from about 150 minutes to about 180 minutes; or from about 180 minutes to about 210 minutes; or from about 210 minutes to about 240 minutes.

The quenching time of the capillary whole blood with the quenching solution can range from about 5 minutes to about 60 minutes; or from about 5 minutes to about 10 minutes; or from about 10 minutes to about 15 minutes; or from about 15 minutes to about 20 minutes; or from about 20 minutes to about 30 minutes; or from about 30 minutes to about 40 minutes; or from about 40 minutes to about 50 minutes; or from about 50 minutes to about 60 minutes.

In several embodiments, agitation can be applied to inactivate aldehyde during the quenching step. In some embodiments, gentle and periodic agitation can be applied to quench aldehyde activity during the quenching step. In certain embodiments, agitation such as stirring, shaking, whisking, blending, and/or folding can be applied manually or via a machine or a device. The periodicity and/or the intensity of the agitation can be optimized to properly inactivate aldehyde activity while preserving the whole blood sample.

The preserved whole blood can be utilized (104) for various downstream applications. In some embodiments, the preserved blood sample is used immediately after quenching (e.g., within 1 hour). In some embodiments, the preserved whole blood is stored for an extended period of time, which can be performed at room temperature. In various embodiments, the preserved whole blood is stored at room temperature for greater than or equal to about 2 hours; the preserved whole blood is stored at room temperature for greater than or equal to about 4 hours; the preserved whole blood is stored at room temperature for greater than or equal to about 6 hours; the preserved whole blood is stored at room temperature for greater than or equal to about 8 hours; the preserved whole blood is stored at room temperature for greater than or equal to about 12 hours; the preserved whole blood is stored at room temperature for greater than or equal to about 16 hours; the preserved whole blood is stored at room temperature for greater than or equal to about 24 hours; the preserved whole blood is stored at room temperature for greater than or equal to about 30 hours; the preserved whole blood is stored at room temperature for greater than or equal to about 36 hours; the preserved whole blood is stored at room temperature for greater than or equal to about 42 hours; the preserved whole blood is stored at room temperature for greater than or equal to about 48 hours; the preserved whole blood is stored at room temperature for greater than or equal to about 72 hours; the preserved whole blood is stored at room temperature for greater than or equal to about 96 hours; the preserved whole blood is stored at room temperature for greater than or equal to about 120 hours; the preserved whole blood is stored at room temperature for greater than or equal to about 144 hours; the preserved whole blood is stored at room temperature for greater than or equal to about 168 hours; or the preserved whole blood is stored for greater than or equal to about 336 hours.

While various processes for preserving whole blood for downstream applications are described above with reference to FIG. 1, any variety of processes that utilize preservation agents to preserve small volume whole blood can be utilized as appropriate to the requirements of specific applications in accordance with various embodiments of the invention. For the purposes of illustrating a specific example of preserving small volume whole blood for single cell applications in accordance with various embodiments of the invention are discussed further below.

### Preservation of Small Volume Whole Blood for Single Cell Applications

Many embodiments implement various preservation processes to fixate and preserve nucleated cells from small volume whole blood for single cell applications. The preservation processes can preserve whole blood and single cells therein for analysis after storage for an extended period of time.

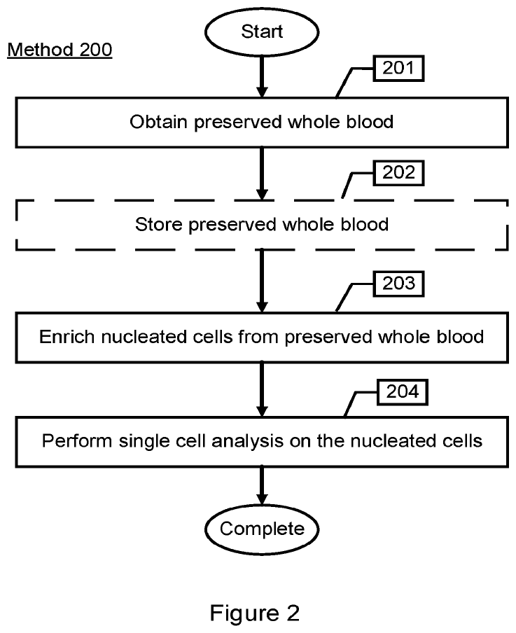

FIG. 2 illustrates a method for preserving whole blood for single cell applications in accordance with an embodiment of the invention. Method 200 can start by obtaining (201) a preserved small volume whole blood sample. The small volume whole blood sample can first be collected using a blood collection method including (but not limited to) capillary blood sampling, venipuncture sampling, and arterial sampling. In some embodiments, the small volume whole blood sample is preserved as described in FIG. 1.

In some embodiments, a sample of small volume whole blood has a volume from about 20 μL to about 1 mL. In various embodiments, a small volume of whole blood is a volume from about 20 μL to about 30 μL; a small volume of whole blood is a volume from about 30 μL to about 40 μL; a small volume of whole blood is a volume from about 40 μL to about 50 μL; a small volume of whole blood is a volume from about 50 μL to about 60 μL; a small volume of whole blood is a volume from about 60 μL to about 70 μL; a small volume of whole blood is a volume from about 70 μL to about 80 μL; a small volume of whole blood is a volume from about 80 μL to about 90 μL; a small volume of whole blood is a volume from about 90 μL to about 100 μL; a small volume of whole blood is a volume from about 100 μL to about 110 μL; a small volume of whole blood is a volume from about 110 μL to about 120 μL; a small volume of whole blood is a volume from about 120 μL to about 130 μL; a small volume of whole blood is a volume from about 130 μL to about 140 μL; a small volume of whole blood is a volume from about 140 μL to about 150 μL; a small volume of whole blood is a volume from about 150 μL to about 200 μL; a small volume of whole blood is a volume from about 200 μL to about 250 μL; a small volume of whole blood is a volume from about 250 μL to about 300 μL; a small volume of whole blood is a volume from about 300 μL to about 350 μL; a small volume of whole blood is a volume from about 350 μL to about 400 μL; a small volume of whole blood is a volume from about 400 μL to about 450 μL; or a small volume of whole blood is a volume from about 450 μL to about 500 μL; or a small volume of whole blood is a volume from about 500 μL to about 550 μL; or a small volume of whole blood is a volume from about 550 μL to about 600 μL; or a small volume of whole blood is a volume from about 600 μL to about 650 μL; or a small volume of whole blood is a volume from about 650 μL to about 700 μL; or a small volume of whole blood is a volume from about 700 μL to about 750 μL; or a small volume of whole blood is a volume from about 750 μL to about 800 μL; or a small volume of whole blood is a volume from about 800 μL to about 850 μL; or a small volume of whole blood is a volume from about 850 μL to about 900 μL; or a small volume of whole blood is a volume from about 900 μL to about 950 μL; or a small volume of whole blood is a volume from about 950 μL to about 1 mL.

The small volume whole blood sample can be preserved with a preservation solution. In some embodiments, the preservation solution comprises a saccharide-epichlorohydrin copolymer and a molecular compound comprising an aldehyde functional group. Fixation of whole blood for the purpose of single cell applications needs the cells in the sample to remain separate from each other. The saccharide-epichlorohydrin copolymer in the fixation solution can reduce cell aggregation when exposed to aldehyde to inhibit cell-to-cell contact to prevent cells from becoming fixed together. Preservation of whole blood for the purpose of single cell RNA sequencing also needs to prevent RNA degradation from RNases (RNA degrading proteins). Aldehyde in the fixation preservation solution can inactivate RNases.

The preservation of a small volume whole blood can be carried out at room temperature. The preservation process can be carried out without heating, or heat treatment, or cooling, or cooling treatment.

A quencher can be added to inactivate the aldehyde. The addition of a quencher can preserve the whole blood without removal of aldehyde from the preserved whole blood solution. The quencher can be a molecular compound comprising an amine (or amino) functional group. The amine functional group can react with the aldehyde group and quench the crosslinking reaction. Examples of molecular compounds comprising an amine functional group include (but are not limited to): tris, tris buffer, glycine, glycine buffer, and any combinations thereof.

In some embodiments, the preserved blood sample is used immediately after quenching (e.g., within 1 hour). In some embodiments, the preserved whole blood can be stored (202) for an extended period of time, which can be performed at room temperature. In various embodiments, the preserved whole blood is stored at room temperature for greater than or equal to about 2 hours; the preserved whole blood is stored at room temperature for greater than or equal to about 4 hours; the preserved whole blood is stored at room temperature for greater than or equal to about 6 hours; the preserved whole blood is stored at room temperature for greater than or equal to about 8 hours; the preserved whole blood is stored at room temperature for greater than or equal to about 12 hours; the preserved whole blood is stored at room temperature for greater than or equal to about 16 hours; the preserved whole blood is stored at room temperature for greater than or equal to about 24 hours; the preserved whole blood is stored at room temperature for greater than or equal to about 30 hours; the preserved whole blood is stored at room temperature for greater than or equal to about 36 hours; the preserved whole blood is stored at room temperature for greater than or equal to about 42 hours; the preserved whole blood is stored at room temperature for greater than or equal to about 48 hours; the preserved whole blood is stored at room temperature for greater than or equal to about 72 hours; the preserved whole blood is stored at room temperature for greater than or equal to about 96 hours; the preserved whole blood is stored at room temperature for greater than or equal to about 120 hours; the preserved whole blood is stored at room temperature for greater than or equal to about 144 hours; the preserved whole blood is stored at room temperature for greater than or equal to about 168 hours; or the preserved whole blood is stored for greater than or equal to about 336 hours.

In several embodiments, the preserved blood sample can be transported via various means including (but not limited to) shipping, delivery, dropping off, picking up by a courier, to a processing facility. The transportation of the blood sample can be carried out at room temperature without heating, or heat treatment, or cooling, or cooling treatment. Transportation can occur right after quenching or after being stored for a period of time at room temperature. At the processing facility, the blood sample can be processed right after being received or can be stored for a period of time at room temperature.

Nucleated cells can be enriched (203) from preserved whole blood. Nucleated cells (e.g., PBMCs, granulocytes) and/or multilobed nuclei cells (e.g., neutrophils) can be isolated and collected from the preserved whole blood. Nucleated cells can be enriched by a number of methodologies, such as (for example) centrifugation, density gradient centrifugation, cell preparation tubes, PBMC isolation tubes (such as SepMate®, Vacutainer®), magnetic cell separation kits (such as EasySep®), immunoprecipitation, immune separation, and column purification. In one example density gradient centrifugation is performed by adding a density gradient buffer to the blood sample. Centrifugation can separate the components by their densities. The plasma can be removed by pipetting. Any remaining debris can be removed by pipetting. The isolated nucleated cells can be collected using pipettes and/or Pasteur pipettes. To further remove any remaining red blood cells, a red blood cell lysis buffer can be added to the enriched nucleated cells. The nucleated cells can then be washed and centrifuged. The isolated and collected nucleated cells can be counted to prepare for single cell applications. In some embodiments, the enriched nucleated cells can be further stored in a freezer or using a cryogenic source (such as liquid nitrogen) for a prolonged period of time. If cryopreserved, the nucleated cells can be thawed when ready to use in downstream applications.

Single cell analysis can be performed (204) on the nucleated cells. Examples of single cell assays that are compatible with the preserved whole blood include (but are not limited to): single cell nucleic acid sequencing, single cell DNA sequencing, single cell genomic analysis, single cell RNA sequencing, single cell transcriptome analysis, single cell gene expression assays, single cell proteomic analysis (e.g., via mass spectrometry), single cell immunodetection assays, single cell metabolomic analysis (e.g., via mass spectrometry), single cell chromatin analysis, single cell methylome sequencing, single cell bisulfite sequencing, single cell immunoprecipitation assays, and single cell chromatin immunoprecipitation sequencing.

In one example, nucleated cells are used for single cell RNA-seq. The preserved and enriched nucleated cells from whole blood are compatible with various single cell RNA-seq platforms including (but not limited to) droplet-based single cell RNA-seq. Examples of droplet-based single cell RNA-seq platforms include (but are not limited to) 10× Genomics®, Chromium from 10× Genomics®, ddSEQ® from Bio-Rad Laboratories, InDrop® from 1CellBio, μEncapsulator® from Dolomite Bio/Blacktrace Holdings, PIPseq® from Fluent BioSciences, Evercode® from Parse Biosciences, and ScaleBio® from Scale Biosciences.

While various processes for preserving whole blood for single cell applications are described above with reference to FIG. 2, any variety of processes that utilize preservation agents to preserve small volume whole blood can be utilized as appropriate to the requirements of specific applications in accordance with various embodiments of the invention.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for.

### Example 1: Capillary Whole Blood Preservation With Formaldehyde and Ficoll®

Ficoll® can be combined with formaldehyde to preserve capillary whole blood in order to preserve and isolate PBMCs for downstream applications. Ficoll® solution can be made by dissolving the Ficoll® powder in PBS. Dissolving Ficoll® in water can result in hemolysis when being added to the formaldehyde fixation solution and blood, probably due to low osmolarity. The combination of Ficoll® and formaldehyde in the preservation solution produces better yields of enriched PBMCs from the preserved whole blood.

In order to keep preserved blood samples at room temperature for at least 24 hours without removal of preservation solution from blood cells, the formaldehyde in the solution is inactivated via a quenching solution at least 4 times the volume of the preservation solution. The quenching solution can include Ficoll®, glycine/tris buffer, additional tris buffer equimolar to the final amount of formaldehyde, in a PBS solution.

Preservation solutions with only aldehyde compounds may be able to fixate capillary whole blood, but robust enrichment of PBMCs after fixation cannot be achieved. Using formaldehyde directly for preservation results in all cells and debris from the whole blood fixed together such that PBMCs could not be recovered and isolated in quantities for high quality analysis.

In one experiment, formaldehyde of various concentrations was added to the capillary whole blood samples at final concentrations of about 1%, of about 2%, and of about 4%. All three experiments showed that formaldehyde fixated all cells and debris from the whole blood fixed together and PBMCs recovery could not be achieved. Following experiments dilute the capillary whole blood samples with PBS prior to the addition of formaldehyde. A 10-times dilution and a 20-times dilution of the whole blood samples with PBS do not improve the PBMCs recovery after formaldehyde fixation.

### Example 2: Cell Preservation With Capillary Whole Blood

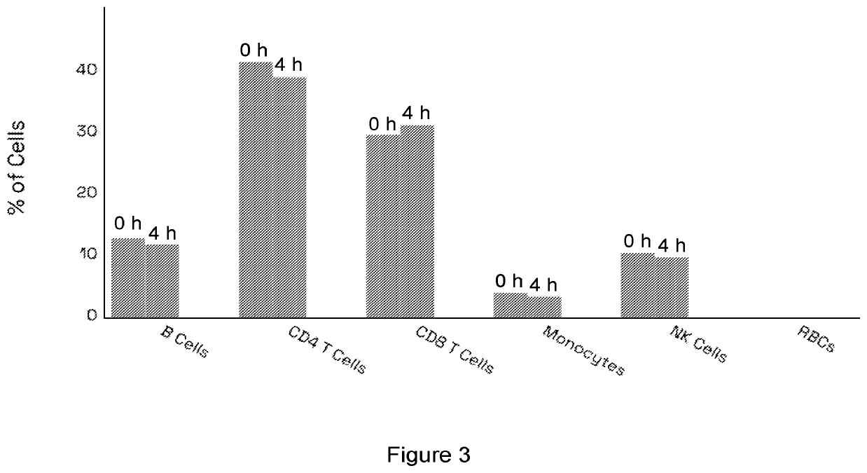

To test the ability to preserve cells within whole blood, capillary whole blood was extracted and then treated in accordance with methods as described herein. Briefly, a solution comprising an aldehyde and a saccharide-epichlorohydrin copolymer was used to preserve capillary whole blood. A glycine solution was utilized to quench the aldehyde reaction. The blood was analyzed immediately (0 hours) and 4 hours after quenching the reaction. After quenching, the whole blood was further processed to perform single-cell RNA sequencing. Known cell-type markers were utilized to identify which blood cells were preserved (FIG. 3). As can be seen in the data results, B cells, CD4 T Cells, CD8 T cells, monocytes, and NK cells each had a similar percentage of cells at 4 hours after preservation as compared with 0 hours. These results suggest that no single cell type was uniquely affected in terms of cell preservation.

### Example 3: Cell Isolation From Capillary Whole Blood After Preservation

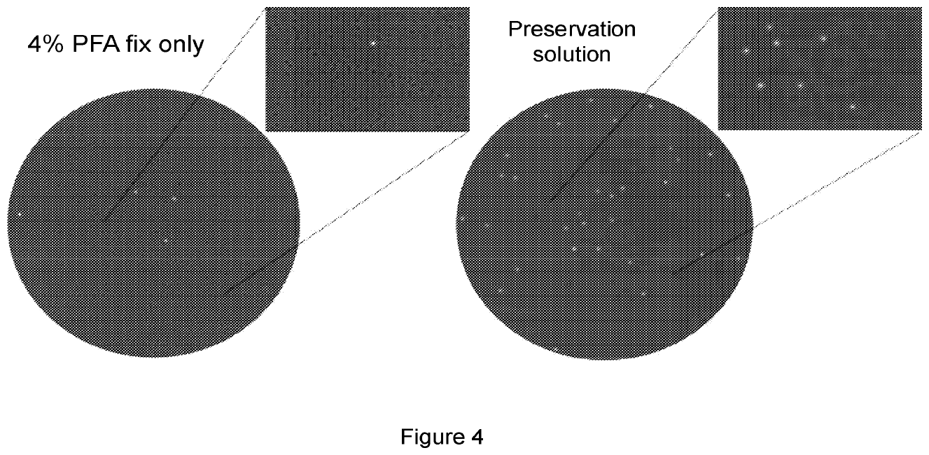

To test the effect of preservation solution on cell isolation from whole blood, capillary whole blood was extracted and then treated in accordance with methods as described herein. Briefly, a solution comprising an aldehyde and a saccharide-epichlorohydrin copolymer was used to preserve capillary whole blood. As a control, cells treated only with aldehyde were also prepared. A glycine solution was utilized to quench the aldehyde reaction. The two preservation techniques were compared for their ability to isolate PBMCs utilizing a commercially available PBMC isolation kit (STEMCELL Technologies, Vancouver, CA) (FIG. 4). As can be seen in the data results, PBMCs were better isolated when preserved using a solution comprising an aldehyde and a saccharide-epichlorohydrin copolymer. These results suggest that a solution comprising an aldehyde and a saccharide-epichlorohydrin copolymer for preservation yields better cell isolation.

### Example 4: Preservation of MALAT1 Gene Expression

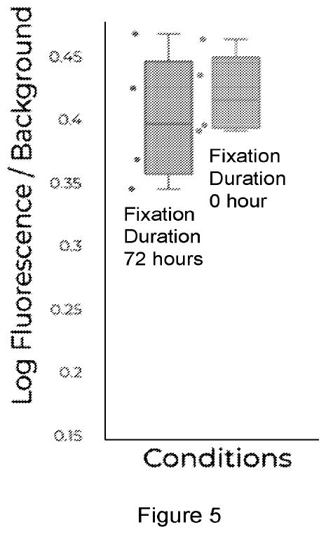

To test the ability to preserve gene expression of cells within whole blood, capillary whole blood was extracted and then treated in accordance with methods as described herein. Briefly, a solution comprising an aldehyde and a saccharide-epichlorohydrin copolymer was used to preserve capillary whole blood. A glycine solution was utilized to quench the aldehyde reaction. MALAT1 gene expression was analyzed immediately (0 hours) and 72 hours after quenching the reaction. After quenching the whole blood was further processed and fluorescence in-situ hybridization using a MALAT1 probe was performed (FIG. 5). As can be seen in the data results, the amount of MALAT1 within the sample preserved for 72 hours was similar to the sample analyzed immediately. These results suggest that the preservation method protects RNA for long periods of storage.

### Example 5: Preservation of RNA Transcripts

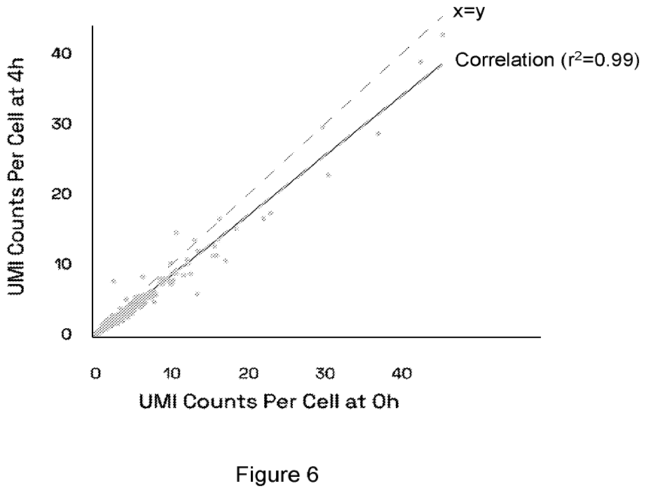

To test the ability to preserve RNA transcripts within whole blood, capillary whole blood was extracted and then treated in accordance with methods as described herein. Briefly, a solution comprising an aldehyde and a saccharide-epichlorohydrin copolymer was used to preserve capillary whole blood. A glycine solution was utilized to quench the aldehyde reaction. The blood was analyzed immediately (0 hours) and 4 hours after quenching the reaction. After quenching, the whole blood was further processed to perform single-cell RNA sequencing. Unique molecular identifiers (UMI) were counted from all cell types to determine the ability to preserve the totality of unique RNA transcripts within single cells (FIG. 6). As can be seen in the data results, the number of UMI between the whole blood analyzed after 4 hours of preservation and analyzed immediately were similar. These results suggest that the cell preservation protocol can preserve the totality of a cell's transcriptome for extended periods of time.

### Example 6: Preservation of RNA Transcripts at Various Time Durations

To test the ability to preserve RNA transcripts within whole blood at different time durations, capillary whole blood was extracted and then treated in accordance with methods as described herein. Briefly, 2 blood samples were taken from 2 participants at the same time. Samples from participant 1 were split into 2 aliquots, and samples from participant 2 were split into 3 aliquots. All samples were fixed immediately after collection. A solution comprising an aldehyde and a saccharide-epichlorohydrin copolymer was used to preserve the whole blood samples. A glycine solution was utilized to quench the aldehyde reaction. Aliquots from participant 1 were kept at room temperature for about 0 hour (0 h sample) and for about 24 hours (24 h sample) after quenching the reaction. Aliquots from participant 2 were kept at room temperature for about 0 hour (0 h sample), for about 24 hours (24 h sample), and for about 72 hours (72 h sample) after quenching the reaction.

After quenching and waiting for various time durations, each aliquot was further processed to perform single-cell RNA sequencing. PBMC isolation was performed to each aliquot. After isolation, each aliquot can be used immediately or can be transferred to −80° C. storage. The samples can be retrieved from −80° C. storage and underwent sample barcoding. The 0 h and 24 h samples were further split into 4 aliquots each. All samples then underwent the 10× Genomics® Flex kit probe hybridization and single-cell sequencing protocol, and were processed on 2 lanes of a 10× Genomics® Chip Q.

DNA libraries were sequenced. The 10× Genomics® Cell Ranger software was used on the resulting FASTQ files to map probe reads onto the associated gene and tally up counts of each gene for each cell and sample barcode. Cells in the resulting gene count matrix were grouped together by performing Leiden clustering on the latent space of a variational autoencoder (scVI) trained to encode the gene count matrix.

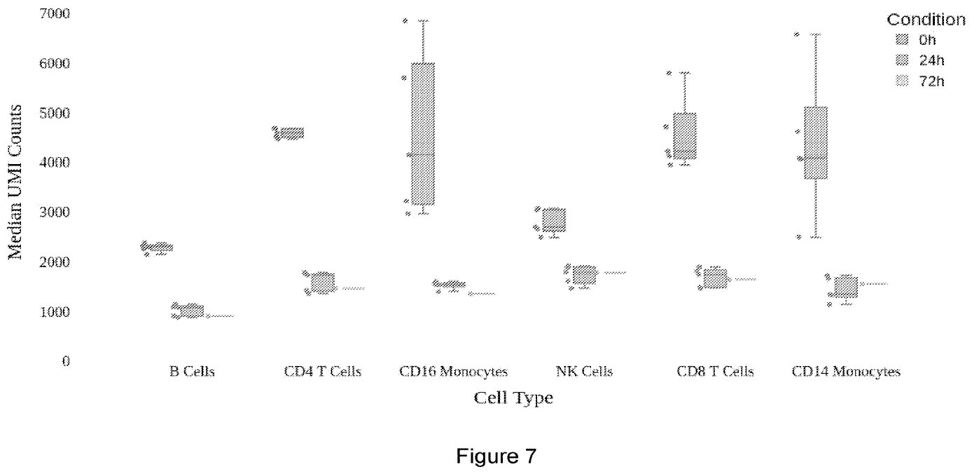

From this data, clusters were assigned to known cell types based on the relative expression of RNA of known cell type marker genes. From these cells assigned to cell types, the total number of UMIs (detected transcripts) can be calculated in each aliquot of each sample. FIG. 7 illustrates median UMI counts of various types of cells from participant 2 samples. UMI were counted from all cell types to determine the ability to preserve the totality of unique RNA transcripts within single cells. FIG. 7 shows an initial drop of UMIs between 0 h and 24 h, but no significant drop of UMIs in the 72 h sample relative to the 24 h sample. This means that RNA degradation or other sources of transcript detection loss stabilize some time before 24 h and are stable between that time point and 72 h, suggesting that 72 h fixed samples can be reliably compared to samples that were fixed for only 24 h.

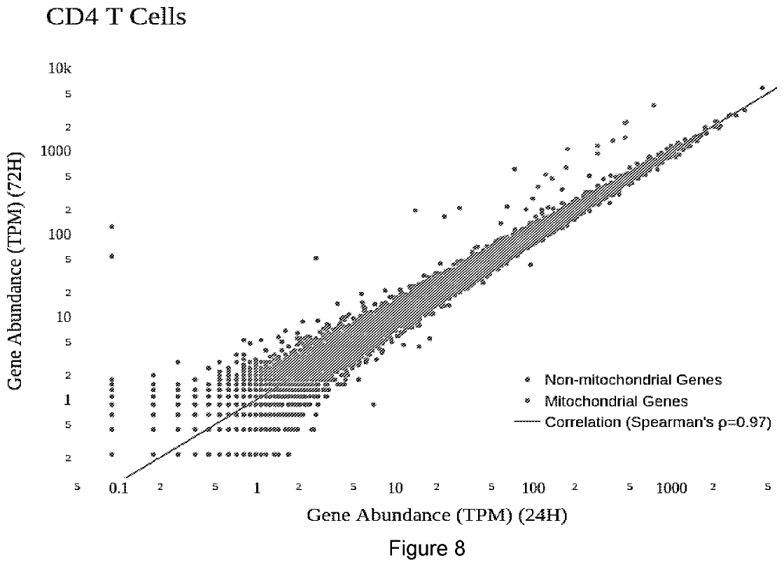

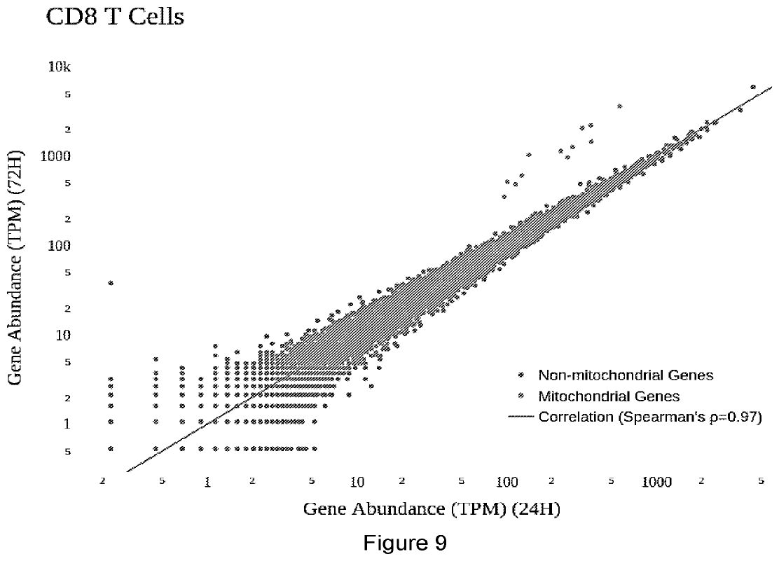

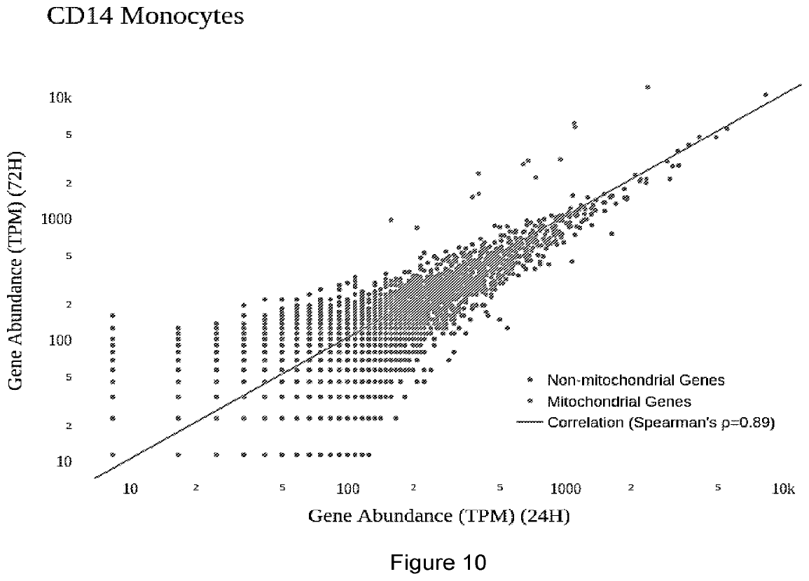

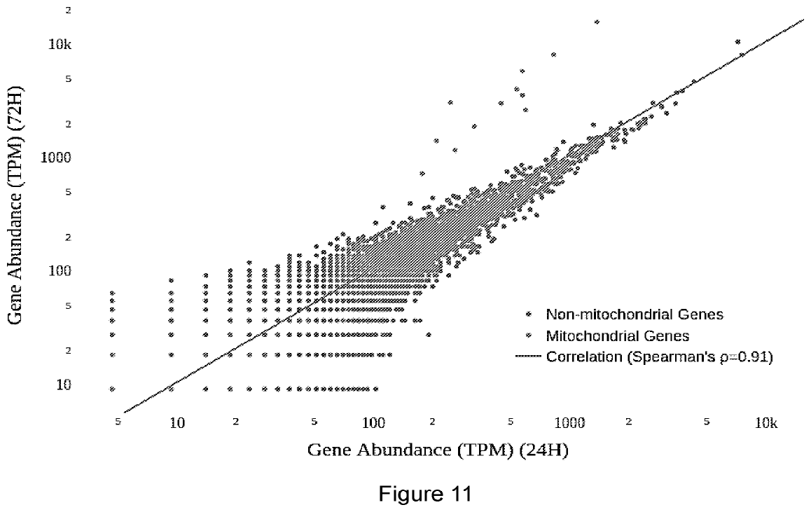

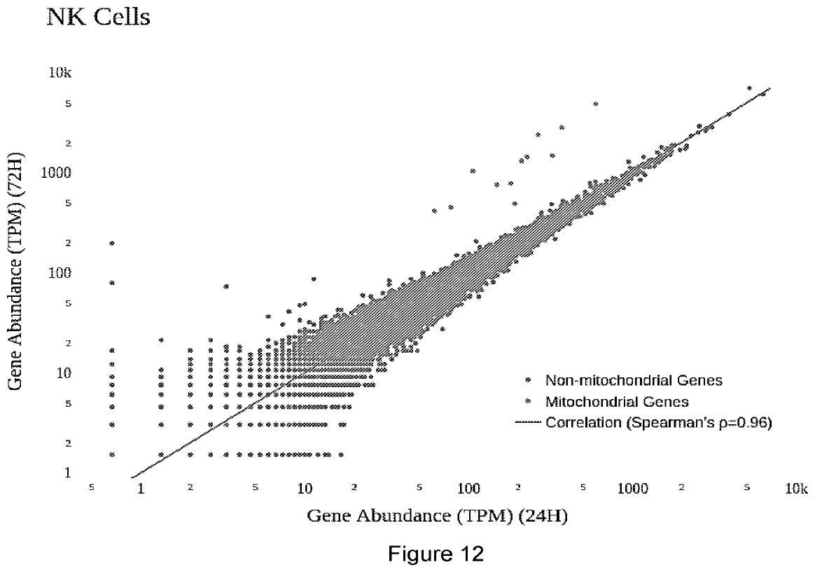

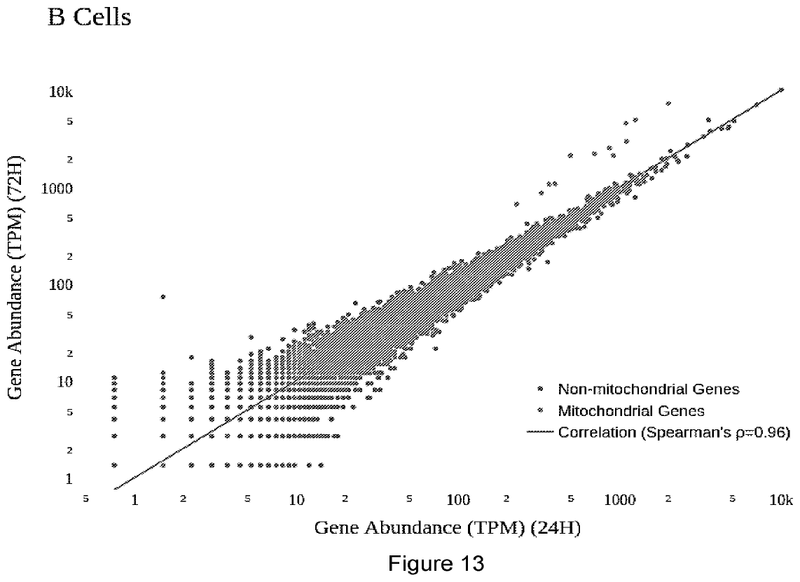

The relative abundances of gene counts are investigated. Some embodiments provide a strong correlation of the cumulative relative abundance of all cells of a type between 24 h and 72 h. FIG. 8 illustrates a gene abundance correlation between the 24 h samples and the 72 h samples for CD4 T cells. The correlation is about 0.97 between the non-mitochondrial genes and the mitochondrial genes. FIG. 9 illustrates a gene abundance correlation between the 24 h samples and the 72 h samples for CD8 T cells. The correlation is about 0.97 between the non-mitochondrial genes and the mitochondrial genes. FIG. 10 illustrates a gene abundance correlation between the 24 h samples and the 72 h samples for CD14 monocytes. The correlation is about 0.89 between the non-mitochondrial genes and the mitochondrial genes. FIG. 11 illustrates a gene abundance correlation between the 24 h samples and the 72 h samples for CD16 monocytes. The correlation is about 0.91 between the non-mitochondrial genes and the mitochondrial genes. FIG. 12 illustrates a gene abundance correlation between the 24 h samples and the 72 h samples for NK cells. The correlation is about 0.96 between the non-mitochondrial genes and the mitochondrial genes. FIG. 13 illustrates a gene abundance correlation between the 24 h samples and the 72 h samples for B cells. The correlation is about 0.96 between the non-mitochondrial genes and the mitochondrial genes. FIGS. 8 through 13 show a strong correlation of the gene abundance between the 24 h samples and the 72 h samples for all the cell types. Thus, 72 h fixed samples are comparable to samples that were fixed for 24 h.

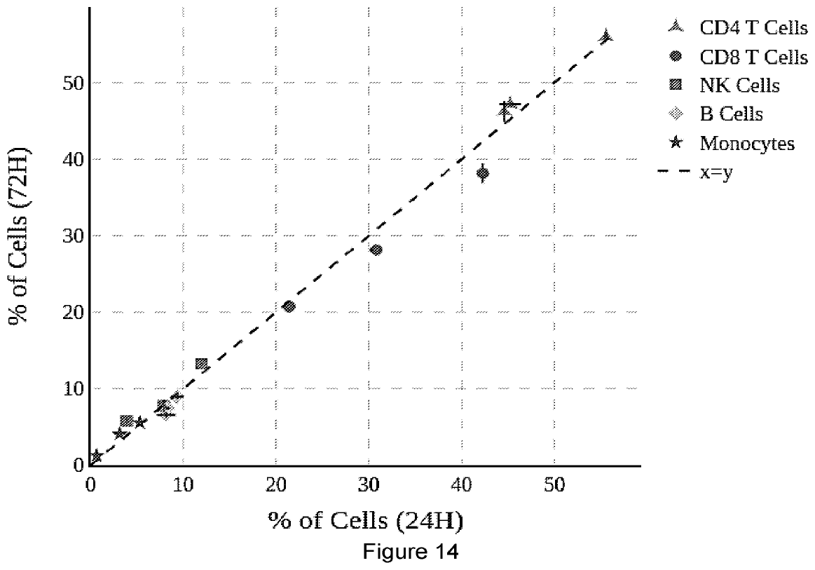

Cell type abundance is further investigated. A subsequent experiment was performed to repeat the fixative protocol with 3 samples for 24 h vs 72 h. Using the computational methods as described above, cells were assigned to their individual cluster. It was validated that cell types maintain their relative abundance between 24 h and 72 h for all participants samples. FIG. 14 illustrates a correlation of percentage of cells between the 24 h samples and the 72 h samples for CD4 T cells, CD8 T cells, NK cells, B cells, and monocytes.

### DOCTRINE OF EQUIVALENTS

As can be inferred from the above discussion, the above-mentioned concepts can be implemented in a variety of arrangements in accordance with embodiments of the invention. Accordingly, although the present invention has been described in certain specific aspects, many additional modifications and variations would be apparent to those skilled in the art. It is therefore to be understood that the present invention may be practiced otherwise than specifically described. Thus, embodiments of the present invention should be considered in all respects as illustrative and not restrictive.

As used herein, the singular terms “a,” “an,” and “the” may include plural referents unless the context clearly dictates otherwise. Reference to an object in the singular is not intended to mean “one and only one” unless explicitly so stated, but rather “one or more.”

As used herein, the terms “approximately,” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

1. A solution for preserving whole blood, comprising:
a saccharide-epichlorohydrin copolymer; and
a molecular compound comprising an aldehyde functional group;
dissolved in a solution.

2. The solution of claim 1, wherein the molecular compound comprising an aldehyde functional group is of: formaldehyde, paraformaldehyde, acetaldehyde, glutaraldehyde, or formalin.

3. The solution of claim 1, wherein the molecular compound comprising an aldehyde functional group has a concentration ranging from 0.5% to 5.0% (% w/v).

4. The solution of claim 1, wherein the saccharide-epichlorohydrin copolymer has a molecular weight from 60,000 Da to 500,000 Da.

5. The solution of claim 1, wherein the saccharide-epichlorohydrin copolymer is sucrose-epichlorohydrin copolymer.

6. The solution of claim 1, wherein the sucrose-epichlorohydrin copolymer is selected from the group consisting of: polysucrose, polysucrose 400, 2-(chloromethyl)oxirane; (2R,3R,4S,5S,6R)-2-[(2S,3S,4S,5R)-3,4-dihydroxy-2,5-bis(hydroxymethyl)oxolan-2-yl]oxy- 6-(hydroxymethyl)oxane-3,4,5-triol, Ficoll®, Ficoll® 70, and Ficoll® 400.

7. The solution of claim 1, wherein the saccharide-epichlorohydrin copolymer has a concentration from 2% to 20% (% w/v).

8. The solution of claim 1, wherein the solution is selected from the group consisting of: distilled water, deionized water, phosphate buffer, phosphate buffer solution, saline, HEPES buffer, MES buffer, MOPS buffer, PIPES buffer, potassium phosphate, sodium phosphate, TAPS, citric acid—sodium citrate buffer, bicarbonate buffer, and any combination thereof.

9. A method to preserve whole blood, comprising:
contacting a whole blood sample with a solution for preserving whole blood, comprising, a saccharide-epichlorohydrin copolymer, and a molecular compound comprising an aldehyde functional group dissolved in a solution; and
adding a quencher to the whole blood solution.

10. The method of claim 9, further comprising:
enriching nucleated cells from the whole blood solution; and
performing a single cell assay on the enriched nucleated cells.

11. The method of claim 9, wherein a ratio (v/v) of the solution to the blood sample is equal to or greater than equal to about 10:1.

12. The method of claim 9, wherein the whole blood sample has a volume from 20 μL to 1 mL.

13. The method of claim 9, wherein the whole blood sample is collected using a capillary blood collection procedure.

14. The method of claim 9, wherein the contacting step is performed at a temperature from 20° C. to 25° C.

15. The method of claim 9, wherein the quencher comprises a molecular compound comprising an amine functional group, dissolved in a solution comprising a sucrose-epichlorohydrin copolymer.

16. The method of claim 15, wherein the molecular compound comprising an amine functional group is provided as a tris buffer, a glycine buffer, or a combination thereof; wherein the tris buffer comprises a tris base selected from the group consisting of: tris(hydroxymethyl)aminomethane, Trizma® base, amino-2-(hydroxymethyl)-1,3-propanediol, 2-,2-Amino-2-(hydroxymethyl)-1,3-propanediol, THAM, tris(hydroxymethyl)aminomethane, and trometamol buffer.

17. The method of claim 15, wherein the quencher has an equivalent or excess molar concentration to the molecular compound comprising the aldehyde functional group.

18. The method of claim 15, wherein the solution further comprises ethylenediaminetetraacetic acid.

19. The method of claims 9, wherein the quencher is provided in a quenching solution in a ratio (v/v) to the solution equal to or greater than 2.

20. The method of claims 9, wherein the adding the quencher step is performed 45 minutes to 80 minutes after the contacting step.

21. The method of claims 9, wherein the quencher comprises a molecular compound comprising an amine functional group, dissolved in a solution comprising sucrose-epichlorohydrin copolymer.

22. The method of claims 9, wherein the quencher is provided in a quenching solution in a ratio (v/v) to the solution equal to or greater than 2.

23. The method of claims 9, further comprising storing the quenched whole blood solution at a temperature from 20° C. to 25° C. for at least 24 hours or at least 72 hours.

24. The method of claim 10, wherein the nucleated cells comprise PBMCs.

25. The method of claim 24, further comprising isolating PBMCs from the quenched whole blood solution using a method selected from the group consisting of: centrifugation, density gradient centrifugation, a cell preparation tube, a PBMC isolation tubes, a magnetic cell separation kit, immunoprecipitation, immune separation, and column purification.

26. The method of claim 24, further comprising storing the isolated PBMCs in a freezer or using a cryogenic liquid.

27. The method of claim 10, wherein a single cell assay comprises single cell RNA sequencing (scRNA-seg).

28-50. (canceled)
